(19) United States
(12) Patent Application Publication
(10) Pub. No.: US 2022/0061468 A1
(43) Pub. Date: Mar. 3, 2022

(54) STRAP ADJUSTMENT DEVICE FOR CONNECTING WITH A BODY OF A MASK

(71) Applicant: QBAS Co., Ltd., Taipei City (TW)

(72) Inventor: Heng-Ea SHIUE, Taipei City (TW)

(21) Appl. No.: 17/211,155

(22) Filed: Mar. 24, 2021

(30) Foreign Application Priority Data
Aug. 31, 2020 (TW) 109211364

(51) Int. Cl. A44B 11/06; A63B 33/00; A44B 11/00

(52) U.S. Cl. CPC A44B 11/06; A44B 11/006; A63B 33/002

(57) ABSTRACT

The present invention provides a strap adjustment device for connecting with a body of a mask. The strap adjustment device comprises a female element, an elastic strap, and a regulating device. The female element is disposed on one side of the body of the mask and has an opening and a slot. The elastic strap has a male element and an adjusting end portion, which is opposite to the male element. The male element has a shape which is substantially corresponding to the female element. The regulating device is connected to the adjusting end portion. When the strap adjustment device is assembled with the body of the mask, the adjusting end portion passes through the opening and the slot before passing through the regulating device in an adjustable manner and being engaged in place by the regulating device. The male element is located in and positioned in the opening.

### CROSS-REFERENCES TO RELATED APPLICATIONS

This application claims the benefit of the Taiwan Patent Application Serial Number 109211364 filed on Aug. 31, 2020, the subject matter of which is incorporated herein by reference.

### BACKGROUND OF THE INVENTION

### Field of the Invention

The present invention relates to a strap adjustment device, which is used to connect to a mask or a body of the mask of diving goggles or similar products, such as goggles, swimming goggles, protective goggles, or safety masks.

### Descriptions of the Related Art

Apparatuses such as diving goggles, swimming goggles, protective goggles, or safety masks currently available on the market are often additionally equipped with an elastic strap to make the body of the mask tit to the face stably, and the elastic strap is most favored by consumers with the design of adjustable tightness. However, the strap pulls the user's hair easily, especially when the regulating element for adjusting tightness of the elastic strap is placed at the joint between two sides of the strap and the body of the mask. In detail, the hair is often accidentally caught in the regulating element when the mechanism of the regulating element is adjusted to open and close. In addition, due to the elasticity of the strap itself, the user's hair often gets tangled with the strap and the regulating element, which is very uncomfortable, especially for users such as women or children with hair of a certain length. Additionally, such a regulating method has the trouble that the left and tight sides need to be adjusted at the same time; otherwise, the strap is easy to be out of balance between the left and right sides, which results in non-uniform tightness of the left and right lens on the face. For people or beginners who do not often use goggles, it takes a lot of tune to adjust the length of the strap, and it is easy for them to lose interest in using or learning water sports. Therefore, the regulating element, which has a shape of a box and is arranged at the center of the strap, has become the best design style of the adjustable strap on the market because it avoids the above problems. Nevertheless, such a design requires a lot of components to be configured, which makes it difficult to reduce the material and assembly costs, and accordingly, the product price cannot be effectively controlled, so it is limited to be used in middle and high-end products only. Details will be further explained below.

Taking swimming goggles as an example, as shown in FIG. 1 and FIG. 2, the conventional goggles mask 1 comprises a body 11 of the mask, two straps 13, two sets of strap fastening components 15, and a regulating element 17, which is arranged in the center of the goggles mask 1. The two straps 13 are respectively coupled to the left and right sides of the body 11 of the mask by two sets of strap fastening components 15, and the other free ends of the two straps 13 are respectively wound out through the regulating element 17. The length of the straps 13 is adjusted by the engage/disengage mechanism of the regulating element 17, Each set of strap fastening components 15 at least comprises components such as a convex buckle 151, a positioning plate 153, a pin 155, and a fixing ring 157. The convex buckle 151 is used to snap connect the strap fastening components 15 to the body 11 of the mask, the elastic strap 13 is fixed by the positioning plate 153 after passing through the pin 155, and the end of the strap 13 is fixed by the fixing ring 157. With such a design of the strap fastening component 15, the components are complicated, the material and mold development costs are quite uneconomical, and the assembly requires more time and labor costs. Therefore, the product price cannot be lowered naturally. Besides, it also increases the cost and troubles of users. For example, if the fastening component made of a rigid material breaks due to long-term use or careless operation, then the whole goggles almost must be discarded, which is extremely uneconomical. In view of this, an urgent need exists in the art to provide a product with greatly reduced components, greatly reduced material and assembly costs, and improved price competitiveness.

### SUMMARY OF THE INVENTION

An objective of the present invention is to provide a strap adjustment device with adjustable tightness for use in goggles, and users can change the tightness of the strap through the adjusting device arranged in the center of the strap, More importantly, through the simplified design of the whole components of the strap adjustment device, not only can the user's hair be prevented from getting tangled with the strap adjustment device, but also the need of the whole material and the assembly cost can be reduced. Therefore, the product price can be beneficial to consumers.

To achieve the aforesaid objectives, the strap adjustment device of the present invention is connected with a body of a mask, and comprises a first female element, a first elastic strap, and a regulating device. The first female element is disposed on a first side of the body of the mask, and the first female element is provided with a first opening and a first slot. The first elastic strap has a first male element and a first adjusting end portion, which is opposite to the first male element, and the first male element has a shape which is substantially corresponding to the first female element. The regulating device is connected to the first adjusting end portion. When the strap adjustment device is assembled with the body of the mask, the first adjusting end portion passes through the first opening and the first slot before passing through the regulating device in an adjustable manner and is adapted to be engaged in place by the regulating device, and the first male element is located in the first female element and is positioned in the first opening.

In an embodiment, the strap adjustment device further comprises a second female element and a second elastic strap. The second female element is disposed on a second side of the body of the mask, and the second female elements provided with a second opening and a second slot. The second elastic strap has a second male element and a second adjusting end portion which is opposite to the second male element, and the second male element has a shape which is substantially corresponding to the second female element. When the strap adjustment device is assembled with the body of the mask, the second adjusting end portion passes through the second opening and the second slot before passing through the regulating device in an adjustable manner and is adapted to be engaged in place by the regulating device, and the second male element is located in the second female element and is positioned in the second opening.

In an embodiment, both the first female element and the second female element are formed of a rigid material, and both the first elastic strap and the second elastic strap are formed of a flexible material. The first elastic strap and the second elastic strap are formed of a material having a Shore A hardness that is substantially not greater than 90. The first female element and the second female element have a substantially same shape and are formed of a substantially same material, and the first elastic strap and the second elastic strap have a substantially same length and are formed of a substantially same material.

In an embodiment, when the first male element is located in the first female element and is positioned in the first opening, the first male element has a distal end which protrudes out of the first opening for a height of at least 0.5 ram, and when the second male element is located in the second female element and is positioned in the second opening, the second male element has a distal end which protrudes out of the second opening for a height of at least 0.5 mm.

In an embodiment, the distal end of the first male element protrudes out of the first opening and has a lateral width that is not smaller than a diameter of the first opening; and the distal end of the second male element protrudes out of the second opening and has a lateral width that is not smaller than a diameter of the second opening.

Moreover, to achieve the aforesaid objective, the strap adjustment device of the present invention is connected with a body of a mask and comprises a first male element, a first elastic strap, and a regulating device. The first male element has a neck portion and a head portion substantially larger than the neck portion, and the neck portion extends from a first side of the body of the mask. The first elastic strap has a first female element and a first adjusting end portion which is opposite to the first female element, and the first female element has a shape which is substantially corresponding to the neck portion. The regulating device is connected to the first adjusting end portion. When the strap adjustment device is assembled with the body of the mask, the first adjusting end portion passes through the regulating device in an adjustable manner and is adapted to be engaged in place by the regulating device, and the first female element surrounds the first male element from the head portion of the first male element and is positioned in the neck portion of the first male element.

In an embodiment, the strap adjustment device further comprises a second male element and a second elastic strap. The second male element has a neck portion and a head portion substantially larger than the neck portion, and the neck portion extends from a second side of the body of the mask. The second elastic strap has a second female element and a second adjusting end portion which is opposite to the second female element, and the second female element has a shape which is substantially corresponding to the neck portion of the second male element. When the strap adjustment device is assembled with the body of the mask, the second adjusting end portion passes through the regulating device in an adjustable manner and is adapted to be engaged in place by the regulating device, and the second female element surrounds the second male element from the head portion of the second male element and is positioned in the neck portion of the second male element.

In an embodiment, the strap adjustment device further comprises a first guiding element and a second guiding element. The first guiding element is disposed on the first side of the body of the mask, adjacent to the first male element, and provided with a first guiding slot. The second guiding element is disposed on the second side of the body of the mask, adjacent to the second male element, and provided with a second guiding slot. When the strap adjustment device is assembled with the body of the mask, the first adjusting end portion first passes through the first guiding slot and then is connected to the regulating device, and the second adjusting end portion also passes through the second guiding slot first and then is connected to the regulating device.

In an embodiment, when the first female element surrounds the first male element from the head portion of the first male element and is positioned in the neck portion of the first male element, the head portion of the first male element protrudes out of the first elastic strap for a height of at least 0.5 mm. When the second female element surrounds the second male element from the head portion of the second male element and is positioned in the neck portion of the second male element, the head portion of the second male element protrudes out of the second elastic strap for a height of at least 0.5 mm.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic perspective view of a conventional goggle mask;

FIG. 2 is a perspective exploded view of a conventional goggle mask;

FIG. 3 is a schematic perspective view showing the connection between a first embodiment of a strap adjustment device of the present invention and a body of a mask;

FIG. 4 is a perspective exploded view showing the connection between the first embodiment of the strap adjustment device of the present invention and the body of the mask;

FIG. 5 is a top view showing the combination of a first female element and a first elastic strap of the first embodiment of the strap adjustment device according to the present invention;

FIG. 6 is a schematic cross-sectional view showing the combination of the first female element and the first elastic strap of the first embodiment of the strap adjustment device according to the present invention taken along a section line AA′ in FIG. 5;

FIG. 7 is a perspective exploded view of a second embodiment of the strap adjustment device of the present invention and a body of a mask;

FIG. 8 is a top view showing the combination of a first male element and a first elastic strap of the second embodiment of the strap adjustment device according to the present invention;

FIG. 9 is a schematic cross-sectional view showing the combination of the first male element and the first elastic strap of the second embodiment of the strap adjustment device according to the present invention taken along a section line BB′ of FIG. 8; and

FIG. 10 is a perspective exploded view of a third embodiment of a strap adjustment device of the present invention and a body of a mask.

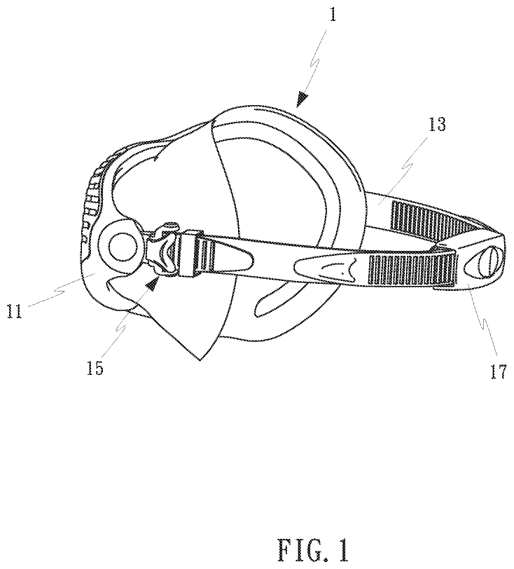

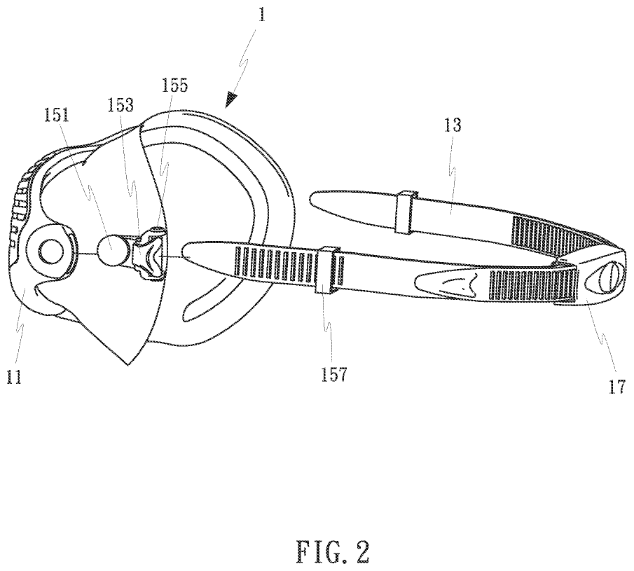

### DESCRIPTION OF THE PREFERRED EMBODIMENT

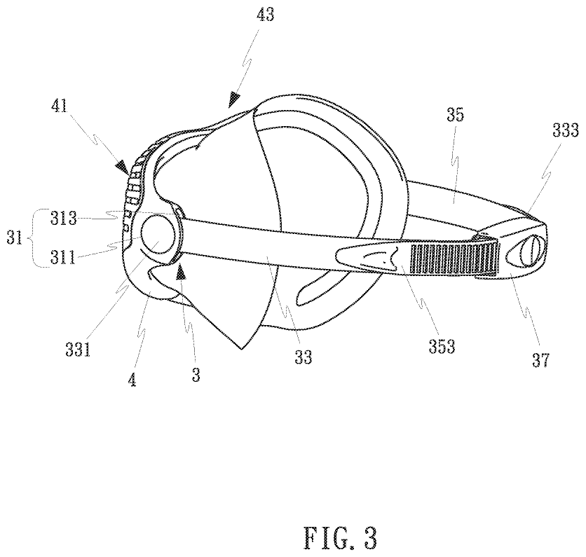

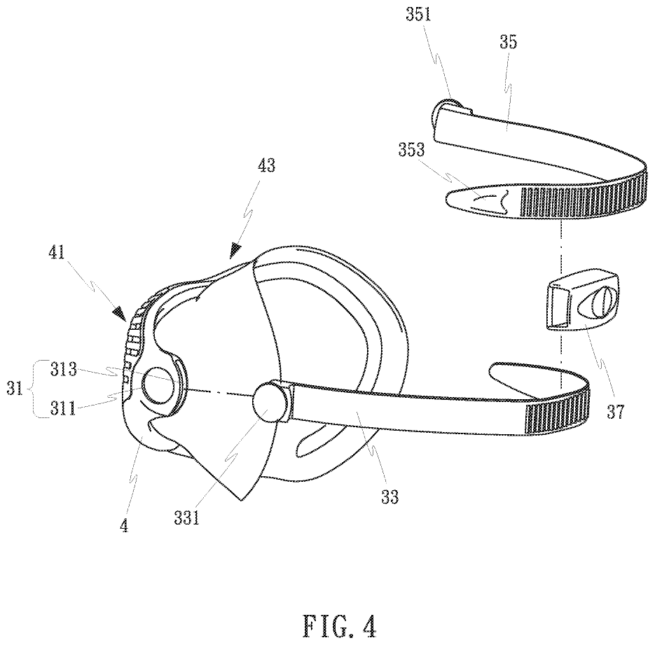

Please refer to FIG. 3 and FIG. 4, which are respectively a schematic perspective view showing the connection between a strap adjustment device 3 and a body 4 of a mask according to the first embodiment of the present invention, and a perspective exploded view before the connection of the strap adjustment device 3 and the body 4 of the mask according to the first embodiment of the present invention. Like the conventional goggle mask structure, the body 4 of the mask has a frame, lenses matching the left and right eyes of a user, and a nose mask covering the nose of the user. Since these basic structures are not the focus of the present invention, such structures will not be further described herein. In this embodiment, when a user wears a goggle mask, the body 4 of the mask elastically and adjustably surrounds the head by using the strap adjustment device 3. The structure of the strap adjustment device 3 is detailed as follows.

In this embodiment, the strap adjustment device 3 comprises a first female element 31, a second female element (not shown), a first elastic strap 33, a second elastic strap 35, and a regulating device 37. The body 4 of the mask has a first side 41 and a second side 43, which are respectively on the left and right sides when the user wears the goggle mask.

The first female element 31 is disposed on the first side 41 of the body 4 of the mask. The first female element 31 is provided with a first opening 311 and a first slot 313. A second female element (not shown) is disposed on the second side 43 of the body 4 of the mask. The second female element is provided with a second opening (not shown) and a second slot (not shown). For convenience of manufacturing, it is preferable that the second female element and the first female element 31 have the same shape and are made of the same material, but they are not limited thereto.

The first elastic strap 33 has a first male element 331 and a first adjusting end portion 333 which is opposite to the first male element 331. That is, the first male element 331 and the first adjusting end portion 333 are respectively located at two ends of the first elastic strap 33. The first male element 331 has a shape substantially corresponding to the first female element 31, and the shape of the distal end of the first adjusting end portion 333 is narrower. Similarly, the second elastic strap 35 has a second male element 351 and a second adjusting end portion 353 which is opposite to the second male element 351. That is, the second male element 351 and the second adjusting end portion 353 are respectively located at both ends of the second elastic strap 35. The second male element 351 has a shape substantially corresponding to the second female element, and the shape of the distal end of the second adjusting end portion 353 is narrower.

The regulating device 37 is connected to the first adjusting end portion 333 and the second adjusting end portion 353.

As shown in FIG. 4 when the strap adjustment device 3 is assembled with the body 4 of the mask, due to the narrower shape of the distal end, the first adjusting end portion 333 of the first elastic strap 33 can easily pass through the first opening 311 of the first female element 31 and the first slot 313 of the first female element 31 before passing through the regulating device 37 in an adjustable manner and is adapted to be engaged in place by the regulating device 37. At this time, because the shape of the first male element 331 substantially corresponds to that of the first female element 31, and depending on the softness and elasticity of the first male element 331, the first male element 331 can just be accommodated and located in the first female element 31 by pulling force and positioned in the first opening 311.

Similarly, due to the narrower shape of the distal end, the second adjusting end portion 353 of the second elastic strap 35 can easily pass through the second opening of the second female element and the second slot of the second female element before passing through the regulating device 37 in an adjustable manner and is adapted to be engaged in place by the regulating device 37. At this time, because the shape of the second male element 351 substantially corresponds to that of the second female element, and depending on the softness and elasticity of the second male element 351, the second male element 351 can just be accommodated and located in the second female element by pulling force and positioned in the second opening.

It shall be noted that, shapes of the distal ends of the first adjusting end portion 333 and the second adjusting end portion 353 are not limited to those described in this embodiment and the corresponding drawings, and other aspects shall be readily inferred by those of ordinary skill in the art.

In order to make the first elastic strap 33 and the second elastic strap 35 pass through the first opening 311 of the first female element 31 and the second opening of the second female element respectively by pulling force, both the first elastic strap 33 and the second elastic strap 35 are made of flexible materials, while both the first female element 31 and the second female element are made of rigid materials. In practical application, the first elastic strap 33 and the second elastic strap 35 are made of a material having a Shore A hardness that is substantially not greater than 90. For example, the materials of the first elastic strap 33 and the second elastic strap 35 are preferably selected from silicone rubber, polyvinyl chloride (PVC), thermoplastic elastomer (PE), thermoplastic rubber (TPR), thermoplastic polyurethane (TPU), rubber, or a combination thereof, but the present invention is not limited thereto, and other soft and elastic materials may also be applied to the first elastic strap and the second elastic strap of the present invention.

In practical operation, the first female element 31 and the second female element which are respectively located on the first side 41 and the second side 43 of the body 4 of the mask have a substantially same shape and are formed of a substantially same material. Similarly, the first elastic strap 33 and the second elastic strap 35 which are respectively located on the first side 41 and the second side 43 of the body 4 of the mask have a substantially same length and are formed of a substantially same material. With the same configuration on the left and right sides, the manufacturing of components and the management of material and parts can be simplified. In addition, the first female element 31 and the second female element of this embodiment are integrally formed with the body 4 of the mask.

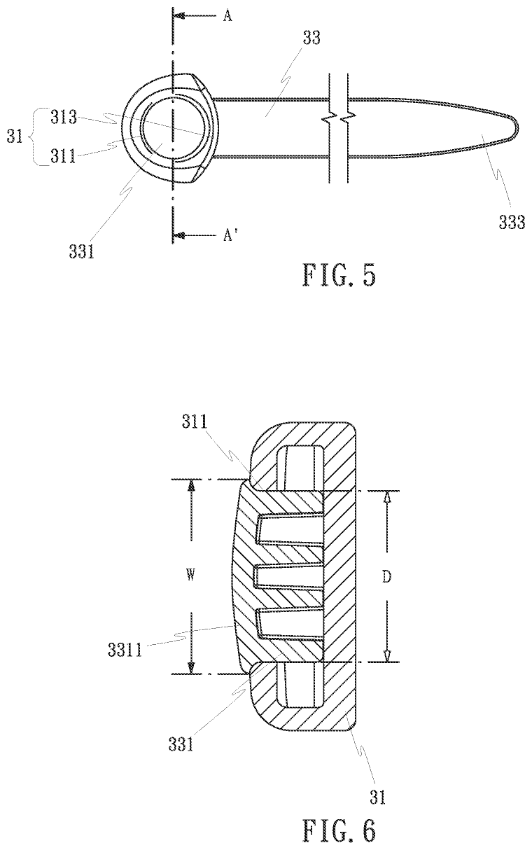

To further describe the connection structure between the first female element 31 and the first elastic strap 33, and the second female element and the second elastic strap 35, please refer to FIG. 5 and FIG. 6, wherein FIG. 5 is a top view showing the combination of the first female element 31 and the first elastic strap 33 of the strap adjustment device 3 of this embodiment, and FIG. 6 is a schematic cross-sectional view showing the combination of the first female element 31 and the first elastic strap 33 of the strap adjustment device 3 of this embodiment taken along the section line AA′ in FIG. 5. Since the connection structure of the second female element and the second elastic strap 35 as well as the connection structure of the first female element 31 and the first elastic strap 33 are the same, only the first female element 31 and the first elastic strap 33 will be exemplified herein.

In detail, when the first male element 331 of the first elastic strap 33 is located in the first female element 31 and is positioned in the first opening 311 of the first female element 31, a distal end 3311 of the first male element 331 protrudes out of the first opening 311 for a height of at least 0.5 mm. Similarly, when the second male element 351 is located in the second female element and is positioned in the second opening, a distal end of the second male element 351 protrudes out of the second opening for a height of at least 0.5 mm. Since the distal end 3311 of the first male element 331 protrudes out of the first opening 311 of the first female element 31, the first male element 331 can be positioned in the first opening 311 without slipping out of the first slot 313. The connection relationship that the distal end of the first male element 351 protrudes out of the second female element is similar to the connection relationship that the distal end 3311 of the first male element 331 protrudes out of the first female element 31, and thus will not be further described herein.

In addition, as shown in FIG. 6, the distal end 3311 of the first male element 331 protrudes out of the first opening 311 of the first female element 31, and has a lateral width W which is greater than a diameter 1) of the first opening 311, and the portion of the first male element 331 which does not protrude out of the first opening 311 of the first female element 31 has a shape that substantially corresponds to the first female element 31. Therefore, as shown in FIG. 6, the first male element 331 of this embodiment is similar to the shape of a mushroom. Therefore, the distal end 3311 of the first male element 331 can achieve the effect of stopping, so that when the first elastic strap 33 and the second elastic strap 35 respectively pass through the first opening 311 of the first female element 31 and the second opening of the second female element by pulling force, the first male element 331 and the second male element 351 will not pass out of t first slot 313 and the second slot respectively due to excessive pulling. Similarly, as can be readily inferred by those of ordinary skill in the art, the distal end of the second male element 351 protrudes out of the second opening of the second female element, and has a lateral width which is also greater than a diameter of the second opening. In another implementation of this embodiment, the distal end 3311 of the first male element 331 protrudes out of the first opening 311 of the first female element 31, and has a lateral width W which may also be equal to a diameter D of the first opening 311. In the assembly process of this implementation, once the first male element 331 and the second male element 351 are positioned in the first opening 311 and the second opening respectively, the pulling force on the first elastic strap 33 and the second elastic strap 35 can be stopped.

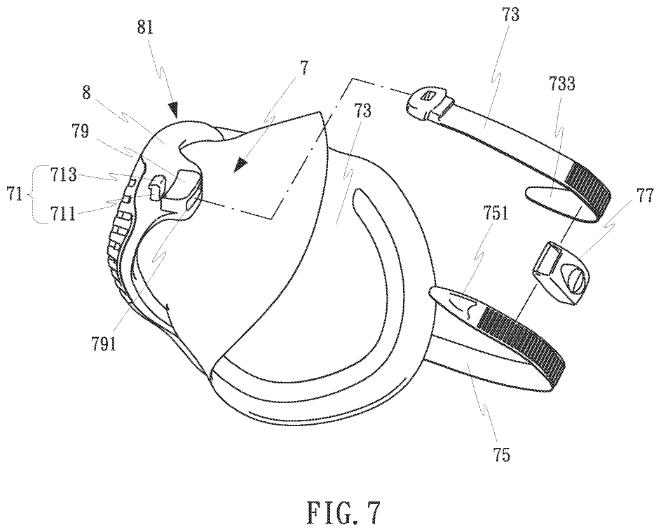

Please refer to FIG. 7, which is a perspective exploded view showing the connection of a strap adjustment device 7 and a body 8 of a mask according to a second embodiment of the present invention. Similar to the first embodiment, the body 8 of the mask has a first side 81 and a second side (not shown), which are respectively on the left and right sides when the user wears the goggle mask. Moreover, the body 8 of the mask is similar to the conventional structure of the goggle mask, which can be readily inferred by those of ordinary skill in the art, and thus the basic structure thereof will not be further described herein.

In this embodiment, the strap adjustment device 7 comprises a first male element 71, a second male element (not shown), a first elastic strap 73, a second elastic strap 75, a regulating device 77, a first guiding element 79, and a second guiding element (not shown).

The first male element 71 has a neck portion 711 and a head portion 713 which is substantially larger than the neck portion 711, and the neck portion 711 extends from a first side 81 of the body 8 of the mask. As shown in the figure, the shape of the first male element 71 in this embodiment is hook-shaped, while in other embodiments, the shape of the first male element 71 may also be mushroom-shaped, inverted cone-shaped or other shapes with a head portion larger than a neck portion.

Similar to the first male element 71, the second male element has a neck portion and a head portion which is substantially larger than the neck portion, and the neck portion extends from a second side of the body 8 of the mask. The shape of the second male element in this embodiment is also hook-shaped, while in other embodiments, the shape of the second male element may be mushroom-shaped, inverted cone-shaped or other shapes with a head portion larger than a neck portion. For convenience of manufacturing, it is preferable that the second male element and the first male element 71 have a same shape and are formed of a same material, but they are not limited thereto.

The first elastic strap 73 has a first female element 731 and a first adjusting end portion 733 which is opposite to the first female element 731, i.e., the first female element 731 and the first adjusting end portion 733 are respectively located at wo ends of the first elastic strap 73. The first female element 731 has a shape which substantially corresponds to the neck portion 711 of the first male element 71, and the shape of the distal end of the first adjusting end portion 733 is narrower.

Likewise, the second elastic strap 75 has a second female element (not shown) and a second adjusting end portion 751 which is opposite to the second female element, i.e., the second female element and the second adjusting end portion 751 are respectively located at two ends of the second elastic strap 75. The second female element has a shape which substantially corresponds to the neck portion of the second male element, and the shape of the distal end of the second adjusting end portion 7M is narrower.

The regulating device 77 is connected to the first adjusting end portion 733 and the second adjusting end portion 751.

As shown in FIG. 7, the first guiding element 79 is disposed on the first side 81 of the body 8 of the mask, adjacent to the first male element 71, and provided with a first guiding slot 791. On the other hand, the second guiding element is disposed on the second side of the body 8 of the mask, adjacent to the second male element, and provided with a second guiding slot.

When the strap adjustment device 7 is assembled with the body 8 of the mask, due to the narrower shape of the distal end, the first adjusting end portion 733 can first readily pass through the first guiding slot 791 before passing through the regulating device 77 in an adjustable manner, and is adapted to be engaged in place by the regulating device 77. Thereafter, depending on the softness and elasticity of the first female element 731, the first female element 731 can surround the first male element 71 from the head portion 713 of the first male element 71 and be positioned in the neck portion 711 of the first male element 71 by pulling force.

Similarly, due to the narrower shape of the distal end, the second adjusting end portion 751 can also first readily pass through the second guiding slot before passing through the regulating device 77 in an adjustable manner, and is adapted to be engaged in place by the regulating device 77. Thereafter, depending on the softness and elasticity of the second female element, the second female element can surround the second male element from the head portion of the second male element and be positioned in the neck portion of the second male element by pulling force.

It shall be additionally noted that, in the above assembly process, the first adjusting end portion 733 of the first elastic strap 73 and the second adjusting end portion 751 of the second elastic strap 75 first respectively pass through the regulating device 77, and then the first female element 731 of the first elastic strap 73 and the second female element of the second elastic strap 75 respectively surround the first male element 71 and the second male element and are positioned on the first male element 71 and the second male element. However, in other assembly processes, the first female element 731 of the first elastic strap 73 and the second female element of the second elastic strap 75 can also first surround the first male element 71 and the second male element and be positioned on the first male element 71 and the second male element, and then the first adjusting end portion 733 of the first elastic strap 73 and the second adjusting end portion 751 of the second elastic strap 75 respectively pass through the regulating device 77.

It shall also be noted that, the shapes of the distal ends of the first adjusting end portion 733 and the second adjusting end portion 751 are not limited to those described in this embodiment and the corresponding drawings, and other aspects shall be readily inferred by those of ordinary skill in the art.

Its order to make the first elastic strap 73 and the second elastic strap 75 surround the first male element 71 and the second male element from the head portion 713 of the first male element 71 and the head portion of the second male element, both the first elastic strap 73 and the second elastic strap 75 are made of flexible materials, while both the first male element 71 and the second male element are made of rigid materials. In practical application, the first elastic strap 73 and the second elastic strap 75 are made of a material having a Shore A hardness that is substantially not greater than 90. For example, the materials of the first elastic strap 73 and the second elastic strap 75 are preferably selected from materials such as silicone rubber, polyvinyl chloride (PVC), thermoplastic elastomer (TPE), thermoplastic rubber (TPR), thermoplastic polyurethane (TPU), rubber, or a combination thereof, but the present invention is not limited thereto, and other soft and elastic materials may also be applied to the first elastic strap and the second elastic strap of the present invention.

In this embodiment, the first guiding element 79 and the second guiding element are also made of rigid materials. Based on the above descriptions, the shapes of the first adjusting end portion 733 of the first elastic strap 73 and the second adjusting end portion 751 of the second elastic strap 75 are adapted to correspond to shapes of the first guiding slot 791 of the first guiding element 79 and the second guiding slot of the second guiding element, respectively. In other embodiments, since the first elastic strap 73 and the second elastic strap 75 are made of flexible materials, the shapes of the first adjusting end portion 733 and the second adjusting end portion 751 may also be slightly larger than the shapes of the first guiding slot 791 and the second guiding slot depending on the elasticity of materials. In another embodiment, the shapes of the first adjusting end portion 733 and the second adjusting end portion 751 may also be smaller than the shapes of the first guiding slot 791 and the second guiding slot.

In practical operation, the first male element 71 and the second male element which are respectively located on the first side 81 and the second side of the body 8 of the mask have a substantially same shape and are formed of a substantially same material. Similarly, the first elastic strap 73 and the second elastic strap 75 which are respectively located on the first side 81 and the second side of the body 8 of the mask have a substantially same length and are formed of a substantially same material. With the same configuration on the left and right sides, the manufacturing of components and the management of material and parts can be simplified.

In this embodiment, the first guiding element 79 and the second guiding element have a substantially same shape and are formed of a substantially same material. Further speaking, the materials of the first guiding element 79 and the second guiding element may be the same as the materials of the first male element 71 and the second male element, which simplifies the preparation of raw materials for the process. In addition, the first male element 71 and the second male element of this embodiment are integrally formed with the body 8 of the mask.

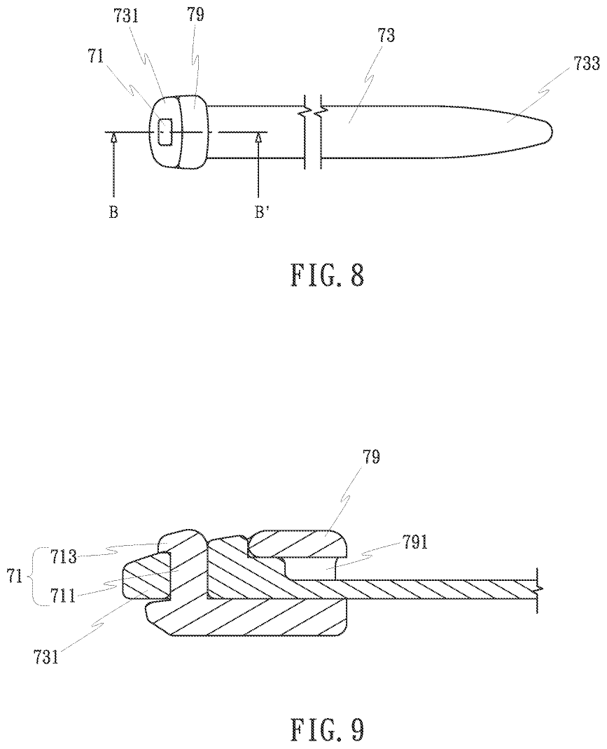

To further describe the connection structure between the first male element 71 and the first elastic strap 73 as well as the connection structure between the second male element and the second elastic strap 75, please refer to FIG. 8 and FIG. 9 together, wherein FIG. 8 is a top view showing the combination of the first male element 71 and the first elastic strap 73 of the strap adjustment device 7 of this embodiment, and FIG. 9 is a schematic cross-sectional view showing the combination of the first male element 71 and the first elastic strap 73 of the strap adjustment device 7 of this embodiment taken along the section line BB′ in FIG. 8, Since the connection structure between the second male element and the second elastic strap 75 as well as the connection structure between the first male element 71 and the first elastic strap 73 are the same, only the first male element 71 and the first elastic strap 73 will be exemplified herein.

In detail, when the first female element 731 of the first elastic strap 73 surrounds the first male element 71 from the head portion 713 of the first male element 71 and is positioned in the neck portion 711 of the first male element 71, the head portion 713 of the first male element 71 protrudes out of the first elastic strap 73 for a height of at least 0.5 mm. Similarly, as shall be readily inferred by those of ordinary skill in the art, when the second female element of the second elastic strap 75 surrounds the second male element from the head portion of the second male element and is positioned in the neck portion of the second male element, the head portion of the second male element protrudes out of the second elastic strap for a height of at least 0.5 mm. In this way, the connection between the male element and the elastic strap can be stabilized.

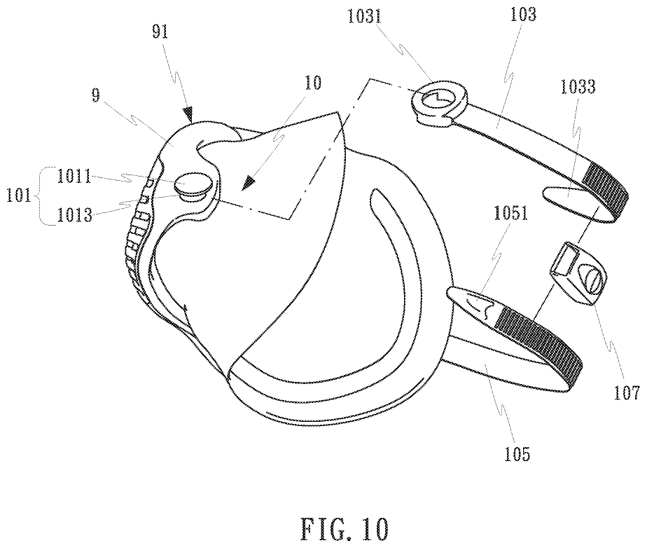

A third embodiment of the present invention is a strap adjustment device 10 for connecting with a body 9 of the mask. The perspective exploded view of the strap adjustment device 10 and the body 9 of the mask is as shown in FIG. 10, Similar to the previous two embodiments, the body 9 of the mask has a first side 91 and a second side (not shown), which are respectively on the left and right sides when the user wears the goggle mask. Moreover, the body 9 of the mask is similar to the conventional structure of the goggle mask, which can be readily interred by those of ordinary skill in the art, and thus the basic structure thereof will not be further described herein.

The strap adjustment device 10 of this embodiment is similar to the strap adjustment device 7 of the second embodiment, and the main difference is that the strap adjustment device 10 omits the guiding element. In detail, the strap adjustment device 10 of this embodiment comprises a first male element 101, a second male element (not shown), a first elastic strap 103, a second elastic strap 105, and a regulating device 107.

The first male element 101 has a neck portion 1013 and a head portion 1011 which is substantially larger than the neck portion 1013, and the neck portion 1013 extends from a first side 91 of the body 9 of the mask. As shown in the figure, the shape of the first male element 101 in this embodiment is mushroom-shaped, while in other embodiments, the shape of the first male element may also be hook-shaped, inverted cone-shaped or other shapes with a head portion larger than a neck portion.

Similar to the first male element 101, the second male element has a neck portion and a head portion which is substantially larger than the neck portion, and the neck portion extends from a second side of the body 9 of the mask. The shape of the second male element in this embodiment is also mushroom-shaped, while in other embodiments, the shape of the second male element may be hook-shaped, inverted cone-shaped or other shapes with a head portion larger than a neck portion. For convenience of manufacturing, it is preferable that the second male element and the first male element 101 have a same shape and are formed of a same material, but they are not limited thereto.

The first elastic strap 103 has a first female element 1031 and a first adjusting end portion 1033 which is opposite to the first female element 1031, i.e., the first female element 1031 and the first adjusting end portion 1033 are respectively located at two ends of the first elastic strap 103. The first female element 1031 has a shape which substantially corresponds to the neck portion 1013 of the first male element 101, and the shape of the distal end of the first adjusting end portion 1033 is narrower.

Likewise, the second elastic strap 105 has a second female element and a second adjusting end portion 1051 which is opposite to the second female element, i.e., the second female element 1031 and the second adjusting end portion 1051 are respectively located at two ends of the second elastic strap 105. The second female element has a shape which substantially corresponds to the neck portion of the second male element, and the shape of the distal end of the second adjusting end portion 1051 is narrower.

The regulating device 107 is connected to the first adjusting end portion 1033 and the second adjusting end portion 1051.

Different from the second embodiment, when the strap adjustment device 10 is assembled with the body 9 of the mask, due to the softness and elasticity of the first female element 1031, the first, female element 1031 can surround the first male element 101 from the head portion 1011 of the first male element 101 and be positioned in the neck portion 1013 of the first male element 101 by pulling force, and then the first female element 1031 passes through the regulating device 107 in an adjustable manner, and is adapted to be engaged in place by the regulating device 107. Similarly, with the softness and elasticity of the second female element, the second female element surrounds the second male element from the head portion of the second male element and is positioned on the neck portion of the second male element by pulling force, and then the second female element passes through the regulating device 107 in an adjustable manner, and is adapted to be engaged in place by the regulating device 107.

Due to the narrower shape of the distal end, it is easier for the first adjusting end portion 1033 and the second adjusting end portion 1051 to pass through the regulating device 107. It shall also be noted that, the shapes of the distal ends of the first adjusting end portion 1033 and the second adjusting end portion 1051 are not limited to those described in this embodiment and the corresponding drawings, and other aspects shall be readily inferred by those of ordinary skill in the art.

It shall be additionally noted that, in the above assembly process, the first female element 1031 of the first elastic strap 103 and the second female element of the second elastic strap 105 first respectively surround the first male element 101 and the second male element and are positioned on the first male element 101 and the second male element, and then the first adjusting end portion 1033 of the first elastic strap 103 and the second adjusting end portion 1051 of the second elastic strap 105 respectively pass through the regulating device 107. However, in other assembly processes, the first adjusting end portion 1033 of the first elastic strap 103 and the second adjusting end portion 1051 of the second elastic strap 105 may also first pass through the regulating device 107 respectively, and then the first female element 1031 of the first elastic strap 103 and the second female element of the second elastic strap 105 respectively surround the first male element 101 and the second male element and are positioned on the first male element 101 and the second male element.

In order to make the first elastic strap 103 and the second elastic strap 105 respective surround the first male element 101 and the second male element from the head portion 1011 of the first male element 101 and the head portion of the second male element by pulling force, both the first elastic strap 103 and the second elastic strap 105 are made of flexible materials, while both the first male element 101 and the second male element are made of rigid materials. In practical application, the first elastic strap 103 and the second elastic strap 105 are made of a material having a Shore A hardness that is substantially not greater than 90. For example, the materials of the first elastic strap 103 and the second elastic strap 105 are preferably selected from materials such as silicone rubber, polyvinyl chloride (PVC), thermoplastic elastomer (TPE), thermoplastic rubber (TPR), thermoplastic polyurethane (TPU), rubber, or a combination thereof, but the present invention is not limited thereto, and other soft and elastic materials may also be applied to the first elastic strap and the second elastic strap of the present invention.

In practical operation, the first male element 101 and the second male element which are respectively located on the first side 91 and the second side of the body 9 of the mask have a substantially same shape and are formed of a substantially same material. Similarly, the first elastic strap 103 and the second elastic strap 105 which are respectively located on the first side 91 and the second side of the body 9 of the mask have a substantially same length and are formed of a substantially same material. With the same configuration on the left and right sides, the manufacturing of components and the management of material and parts can be simplified. Moreover, the first male element 101 and the second male element of this embodiment are formed integrally with the body 9 of the mask.

Similarly to the second embodiment, when the first female element 1031 of the first elastic strap 103 surrounds the first male element 101 from the head portion 1011 of the first male element 101 and is positioned in the neck portion 1013 of the first male element 101, the head portion 1011 of the first male element 101 protrudes out of the first elastic strap 103 for a height of at least 0.5 mm. Similarly, as shall be readily inferred by those of ordinary skill in the art, when the second female element of the second elastic strap 105 surrounds the second male element from the head portion of the second male element and is positioned in the neck portion of the second male element, the head portion of the second male element protrudes out of the second elastic strap for a height of at least 0.5 mm. In this way, the connection between the male element and the elastic strap can be stabilized.

According to the above descriptions, the present invention achieves the effect of simplifying the whole components of the strap adjustment device through the shape correspondence and opposite material hardness between the elastic strap and the female element or the male element arranged on the side of the body of the mask. Moreover, in the assembly process, it is unnecessary to put the strap through strap fastening components such as the convex buckle, the positioning plate, the pin, and the fixing ring one by one and then fix it on the regulating element like the case in the conventional goggle mask. In this way, the whole assembly process is simple, and the material, mold, and assembly costs of the product are reduced, thereby providing users with products of a higher cost performance, and improving the competitiveness of the products in the industry.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A strap adjustment device for connecting with a body of a mask, the strap adjustment device comprising:
a first female element, being disposed on a first side of the body of the mask and having a first opening and a first slot;
a first elastic strap, having a first male element and a first adjusting end portion, which is opposite to the first male element, the first male element having a shape which is substantially corresponding to the first female element; and
a regulating device, being connected to the first adjusting end portion;
wherein when the strap adjustment device is assembled with the body of the mask, the first adjusting end portion passes through the first opening and the slot before passing through the regulating device in an adjustable manner and is engaged in place by the regulating device, and the first male element is located in the first female element and is positioned in the first opening.

2. The strap adjustment device as claimed in claim 1, further comprising:
a second female element, being disposed on a second side of the body of the mask and having a second opening and a second slot; and
a second elastic strap, having a second male element and a second adjusting end portion, which is opposite to the second male element, the second male element having a shape which is substantially corresponding to the second female element;
wherein when the strap adjustment device is assembled with the body of the mask, the second adjusting end portion passes through the second opening and the slot before passing through the regulating device in an adjustable manner and is engaged in place by the regulating device, and the second male element is located in the second female element and is positioned in the second opening.

3. The strap adjustment device as claimed in claim 2, wherein the first female element and the second female element are formed of a rigid material, and the first elastic strap and the second elastic strap are formed of a flexible material.

4. The strap adjustment device as claimed in claim 3, wherein the first elastic strap and the second elastic strap are formed of a material having a Shore A hardness of less than 90.

5. The strap adjustment device as claimed in claim 4, wherein the first female element and the second female element have a substantially same shape and are formed of a substantially same material, and the first elastic strap and the second elastic strap have a substantially same length and are formed of a substantially same material.

6. The strap adjustment device as claimed in claim 5, wherein the material of each of the first elastic strap and the second elastic strap is selected from silicone rubber, polyvinyl chloride (PVC), thermoplastic elastomer (TPE), thermoplastic rubber (TPR), thermoplastic polyurethane (TPU), rubber, or a combination thereof.

7. The strap adjustment device as claimed in claim 6, wherein when the first male element is located in the first female element and is positioned in the first opening, the first male element having a distal end which protrudes out of the first opening for a height of at least 0.5 mm, and when the second male element is located in the second female element and is positioned in the second opening, the second male element having a distal end which protrudes out of the second opening for a height of at least 0.5 mm.

8. The strap adjustment device as claimed in claim 7, wherein the distal end of the first male element protrudes out of the first opening and has a lateral width being not smaller than a diameter of the first opening, and the distal end of the second male element protrudes out of the second opening and has a lateral width being not smaller than a diameter of the second opening.

9. The strap adjustment device as claimed in claim 2, wherein the first female element, the second female element, and the body of the mask are integrally formed.

10. A strap adjustment device for connecting with a body of a mask, the strap adjustment device comprising:
a first male element, having a neck portion and a head portion substantially larger than the neck portion, the neck portion extending from a first side of the body of the mask;
a first elastic strap, having a first female element and a first adjusting end portion, which is opposite to the first female element, the first female element having a shape which is substantially corresponding to the neck portion; and
a regulating device, being connected to the first adjusting end portion;
wherein when the strap adjustment device is assembled with the body of the mask, the first adjusting end portion passes through the regulating device in an adjustable manner and is engaged in place by the regulating device, and the first female element surrounds the first male element from the head portion of the first male element and is positioned in the neck portion of the first male element.

11. The strap adjustment device as claimed in claim 10, further comprising:
a second male element; having a neck portion and a head portion substantially larger than the neck portion, the neck portion extending from a second side of the body of the mask; and
a second elastic strap; having a second female element and a second adjusting end portion, which is opposite to the second female element, the second female element having a shape which is substantially corresponding to the neck portion of the second male element;
wherein when the strap adjustment device is assembled with the body of the mask, the second adjusting end portion passes through the regulating device in an adjustable manner and is engaged in place by the regulating device, and the second female element surrounds the second finale element from the head portion of the second male element and is positioned in the neck portion of the second male element.

12. The strap adjustment device as claimed in claim 11, further comprising a first guiding element and a second guiding element, the first guiding element being disposed on the first side of the body of the mask, adjacent to the first male element, and provided with a first guiding slot, the second guiding element being disposed on the second side of the body of the mask, adjacent to the second male element, and provided with a second guiding slot, wherein when the strap adjustment device is assembled with the body of the mask, the first adjusting end portion passes through the first guiding slot and then is connected to the regulating device, and the second adjusting end portion passes through the second guiding slot and then is connected to the regulating device.

13. The strap adjustment device as claimed in claim 12, wherein the first male element and the second male element are formed of a rigid material, and the first elastic strap and the second elastic strap are formed of a flexible material.

14. The strap adjustment device as claimed in claim 13, wherein the first elastic strap and the second elastic strap are formed of a material having a Shore A hardness of less than 90.

15. The strap adjustment device as claimed in claim 14, wherein the first male element and the second male element have a substantially same shape and are formed of a substantially same material, and the first elastic strap and the second elastic strap have a substantially same length and are formed of a substantially same material.

16. The strap adjustment device as claimed in claim 15, wherein the material of each of the first elastic strap and the second elastic strap is selected from silicone rubber, polyvinyl chloride (PVC), thermoplastic elastomer (TPE), thermoplastic rubber (TPR), thermoplastic polyurethane (TPU), rubber, or a combination thereof.

17. The strap adjustment device as claimed in claim 16, wherein when the first female element surrounds the first male element from the head portion of the first male element and is positioned in the neck portion of the first male element, the head portion of the first male element protrudes out of the first elastic strap for a height of at least 0.5 mm, and when the second female element surrounds the second male element from the head portion of the second male element and is positioned in the neck portion of the second male element, the head portion of the second male element protrudes out of the second elastic strap for a height of at least 0.5 mm.

18. The strap adjustment device as claimed in claim 17, wherein the first male element, the second male element, and the body of the mask are integrally formed.
